(19) United States
(12) Patent Application Publication
(10) Pub. No.: US 2017/0143661 A1
(43) Pub. Date: May 25, 2017

(54) METHOD FOR REDUCING THE VIRAL LOAD IN POULTRY CHALLENGED WITH NEWCASTLE DISEASE (ND)/ AVIAN INFLUENZA (AI)

(71) Applicants: Rajalekshmi Mukkalil, Cochin (IN); Haridasan Chirakkal, Chennai (IN); Jayaraman Sathishkumar, Chennai (IN)

(72) Inventors: Rajalekshmi Mukkalil, Cochin (IN); Haridasan Chirakkal, Chennai (IN); Jayaraman Sathishkumar, Chennai (IN)

(21) Appl. No.: 15/331,184

(22) Filed: Oct. 21, 2016

(30) Foreign Application Priority Data
Oct. 23, 2015 (IN) 3429DEL2015

(51) Int. Cl. A61K 31/28; A61K 9/00

(52) U.S. Cl. CPC A61K 31/28; A61K 9/0053

(57) ABSTRACT

A method of reducing viral load in poultry under viral challenge comprising the supplementation of the poultry with an efficacious dosage of a composition comprising a chromium (III) salt of a carboxylic acid.

### CLAIM OF PRIORITY

This application claims priority to Indian Patent Application No. 3429/DEL/2015, filed Oct. 23, 2015, which is incorporated herein by this reference.

### BACKGROUND OF THE INVENTION

The present invention relates generally to poultry health and, more specifically, to a method of reducing viral load in poultry challenged with Newcastle disease (ND)/Avian influenza (AI) by the supplementation of a composition comprising trivalent chromium salts of short chain fatty acids.

Newcastle disease, Low pathogenic avian influenza (LPAI) and highly pathogenic avian influenza (HPAI) are considered most important poultry diseases in terms of the number of livestock units lost for poultry species¹. Newcastle disease is caused by the virulent strains of Avian paramyxovirus serotype-1 (APMV-1) (OIE, 2012). AI is caused by type A Influenza virus of the family Orthomyxoviridae². Avian influenza virus cause systemic or non-systemic infection. The systemic disease is known as Highly Pathogenic Avian Influenza (HPAI) and is characterized by extensive viral replication in vital organs and death within a few days from the onset of clinical signs in the majority of infected animals. The non-systemic form is characterized by mild respiratory and enteric signs and is known as Low Pathogenicity Avian Influenza (LPAI).

So far the major control measure for these diseases is vaccination. The constant evolution and antigenic change of viruses cause the vaccine to become less effective in giving protection. A few compounds have been tested for their antiviral activity against these viruses. Talactac et al.³ studied the effect of high molecular weight poly gamma glutamate against Newcastle disease virus in murine macrophage cells. Gonzalez et al.⁴ reported the in vitro antiviral activity of fucoidan from Cladosiphon okamuranus against NDV. Ueda et al.⁵ studied the effect of persimmon (Diospyros kaki), against NDV and AI viruses, and observed that the tannin extracts inhibit these viruses.

### SUMMARY OF THE INVENTION

The present invention consists of the dietary supplementation of a trivalent chromium salt of short chain fatty acids for reducing the viral load in poultry challenged with Newcastle disease virus and avian influenza.

In some embodiments, the present invention is a method of reducing viral load in poultry under viral challenge by supplementing the poultry with an efficacious dosage of a composition comprising a chromium (III) salt of a carboxylic acid.

In some embodiments, the present invention is a method of reducing viral load in poultry under viral challenge by supplementing the poultry with an efficacious dosage of a composition, wherein the composition forms low levels of Cr+6 and said low levels of Cr+6 in said food composition having the equivalent of less than 8.3 parts per billion of Cr+6 per 8% total chromium in the food composition.

In some embodiments, the present invention is a method wherein the carboxylic acid is selected from the group consisting of short chain fatty acids, specifically including but not limited to propionic acid.

In some embodiments, the present invention is a method of reducing viral load in poultry under viral challenge by supplementing the poultry with an efficacious dosage of a composition, wherein the viral challenge includes, but is not limited to, Newcastle disease, HPAI and LPAI.

In some embodiments, the present invention is a method of reducing viral load in poultry under viral challenge by supplementing the poultry with an efficacious dosage of a composition, wherein the supplementation is through oral route.

In some embodiments, the present invention is a method of reducing viral load in poultry under viral challenge by supplementing the poultry with an efficacious dosage of a composition, wherein the efficacious dosage varies from 400 to 3000 ppm and preferably from 600 to 1500 ppm.

In some embodiments, the present invention is a method of reducing viral load in poultry under viral challenge by supplementing the poultry with an efficacious dosage of a composition, wherein reduction in viral load results in prevention of infection or reduction in clinical symptoms and mortality.

In some embodiments, the present invention is a method of reducing viral load in poultry under viral challenge by supplementing the poultry with an efficacious dosage of a composition, wherein reduction in viral load results in prevention or control of infection, wherein the prevention or control of infection results in enhanced health, performance and survival.

### BRIEF DESCRIPTION OF THE DRAWINGS

FIG. 1 is a chart of serum antibody titers as measured by ELISA on various days post vaccination and challenge. The ELISA titer results are expressed as mean+SE. One way ANOVA and Bonferroni Post test (P<0.05). (n=14, for Unvacc with and without supplementation n=10 birds). Pre-Vac-Pre vaccination.

FIG. 2 is a chart of serum antibody titers as measured by HI on various days post vaccination and challenge. The results are expressed as mean+SE. One way ANOVA and Bonferroni Post test (P<0.05). (n=14, for Unvacc with and without supplementation n=10 birds). Pre-Vac-Pre vaccination.

FIG. 3 is a chart of survival rate in percentage in the various groups post challenge.

FIG. 4 is a chart of the mean viral titer in various organs post-challenge; results are expressed as mean+SE.

FIG. 5 is a chart of the serum interferon gamma levels measured on various days post vaccination. The results are expressed as mean+SE. One way ANOVA and Bonferroni Post test (P<0.05).

FIG. 6 is a chart of the serum Interleukin-6 (IL-6) levels measured on various days post vaccination. The results are expressed as mean+SE. One way ANOVA and Bonferroni Post test (P<0.05).

### DETAILED DESCRIPTION OF PREFERRED EMBODIMENTS

Trivalent chromium (Cr(III)) ion is an essential nutrient in trace amounts. Hexavalent chromium (Cr⁺⁶) is toxic and carcinogenic. It is, therefore, important that supplementation of chromium be in the form of Cr(III) with very small amounts, or preferably none, of Cr⁺⁶. In preferred embodiments, the chromium composition forms low levels of Cr⁺⁶ and said low levels of Cr⁺⁶ in said food composition having the equivalent of less than 8.3 parts per billion of Cr⁺⁶ per 8% total chromium in the food composition.

A carboxylic acid is an acid that contains a carboxyl group (C(═O)OH). The general formula of a carboxylic acid is R—COOH, wherein R refers to the remaining part of the molecule. Short chain fatty acids are carboxylic acids with R being an aliphatic tail having six carbon atoms or fewer. Short chain fatty acids comprise formic, acetic, propionic, butyric, isobutyric, valeric and isovaleric acids.

This invention relates to an organic chromium supplement having salts of Cr(III) and carboxylic acids, preferably short chain fatty acids, including but not limited to chromium propionate. In the case of chromium propionate, supplements having chromium formed as an acid salt from propionic acid (CH₃CH₂COOH), having the structure of Cr(CH₃CH₂COOH)₃. The invention provides a chromium supplement formed as chromium propionate which when fed to animals can be used to effectively reduce the viral load in poultry under a viral challenge.

Methods of making chromium carboxylates are known. One preferred example is the process taught in U.S. Pat. No. 5,591,878, which is incorporated herein by this reference.

Oral administration of the chromium is preferred for many of the applications listed herein because of its ease. Alternatively, chromium propionate can be injected in the parental tissues or into the bloodstream or the organs or tissues or muscle of the mammal or bird directly. Additionally, the supplement can be formulated as an implant for in vivo use or the supplement can be injected into the body cavities or the gastrointestinal tract. This supplement is capable of being supplied as a diet drink or in a powdered form or in a carrier material or with appropriate diluents. This material can be formulated as a tablet or a gel or a pill or the like. The most preferred method of administration of the material is through the addition to food or drinking water.

When fed as a food supplement, the amount of chromium added to the diet is between about 400 parts per million (ppm) and about 3,000 ppm of the diet, with a preferred range of between about 600 ppm and about 1,500 ppm, including all values between such limits, including, for example, without limitation or exception, 502 ppm, 1,237 ppm, 1,911 ppm, 2,170 ppm, 2,573 ppm and 2,999 ppm. Stated another way, in preferred embodiments of the invention, the dosage or efficacious amount can take any value “abcd” ppm wherein a is selected from the numerals 0, 1, 2 and 3, b is selected from the numerals 4, 5, 6, 7, 8 and 9 when a is 0, and in all other cases b, c and d are each individually selected from the numerals 0, 1, 2, 3, 4, 5, 6, 7, 8 and 9. These ranges can be used to determine acceptable amounts of chromium for supplementation through other forms of administration.

Viral infections in poultry result in the presence of the subject virus in the blood, organs and other tissues of the poultry. In the present invention, the virus infections include those which cause Newcastle disease, highly pathogenic avian influenza (HPA1) and low pathogenic avian influenza (LPA1). Supplementation of poultry diets with compositions of the present invention significantly reduce the viral titer and significantly increase the serum profile of cytokines, including IFN-γ and IL-6, in the treated poultry. Supplementation of poultry diets with compositions of the present invention resulted in the prevention of infection and/or the reduction in clinical symptoms and mortality of the treated poultry, with the effect of enhancing the health, performance and survival of the treated poultry.

“Efficacious amount” for the purposes of this application is defined to be the amount of a compound or composition or derivatives thereof of the present invention is an amount that, when administered to a subject, will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the impairment, and the therapeutics or combination of therapeutics selected for administration, and the mode of administration. The skilled worker can readily determine the effective amount for a given situation by routine experimentation.

### EXAMPLE

A study was conducted to investigate the effect of chromium propionate during viral infection (Newcastle disease virus, NDV) under controlled experimental challenge conditions. The study was conducted in broiler birds hatched from SPF eggs. The birds were individually identified by wing tags and sorted into groups. Birds were kept in an isolator until the end of the experimental period. Commercially available live lentogenic NDV vaccine (LaSota strain) was used in this study. The vaccine was delivered by the occulo-nasal route to specific groups on the 5^{th} day of age. NDV2K35/CH/TN/2003 −a genotype VII virus with the whole genome information available under the GenBank accession number KF740478) was propagated in chickens and freeze dried. The EID₅₀ of the vaccine and also the challenge virus were determined in SPF eggs as per standard protocol.

A known titer of the virus was used for vaccination (3×10⁶ EID50/dose) and also for challenge. Birds were challenged with 106 EID 50 titer of the ND challenge virus strain evenly by the intranasal route, on 21st day of age. Birds of specific treatment groups supplemented with chromium propionate, (0.2 mg in 1 ml distilled water) via oral gavage from day 1. Other groups were given same amount of distilled water.

Treatment groups

1. Unvaccinated, challenged, unsupplemented

2. Unvaccinated, challenged, supplemented with chromium propionate

3. Vaccinated, challenged, unsupplemented

4. Vaccinated, challenged, supplemented with chromium propionate

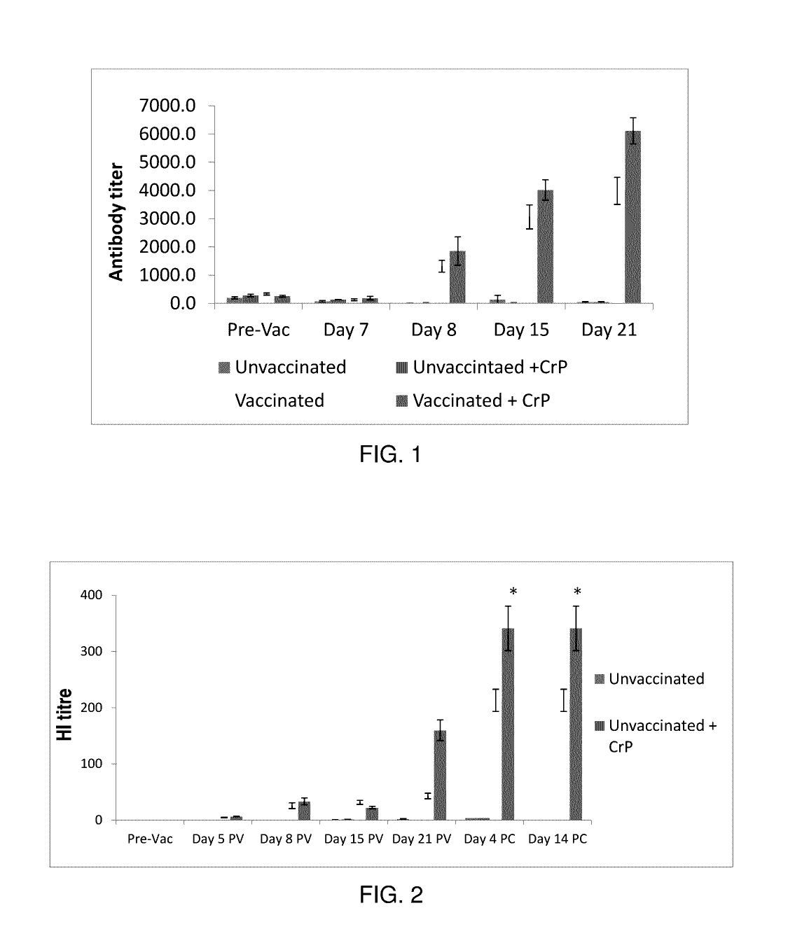

The study showed that the serum antibody titers measured as per ELISA and Haemagglutination Inhibition test (HI) both, were enhanced significantly in the vaccinated, chromium propionate supplemented group, as compared to the respective control (FIG. 1 and FIG. 2).

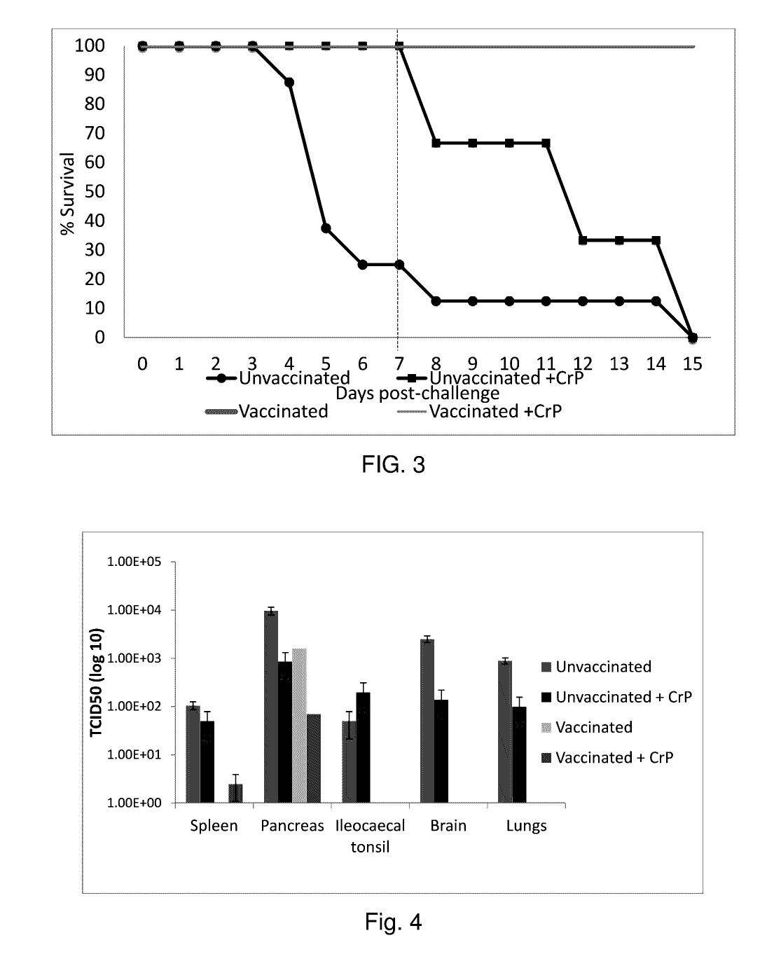

The birds were monitored for 15 days post-challenge and the day-wise mortality was recorded. All birds in the unvaccinated groups exhibited clinical symptoms of ND 5-6 days post-challenge and 100% mortality by day 15. In the unvaccinated chromium propionate supplemented group, the onset of mortality was delayed and daily mortality percentages were lower (P<0.05) as compared to the corresponding control group (FIG. 3).

Viral load in various organs post-challenge was measured. The tissue samples were homogenized, treated with antibiotics and the titration was performed by infecting primary chick embryo fibroblast cells. The cells were fixed 72 to 96 hours after infection and stained with Giemsa stain and the CPEs scored. The TCID₅₀ for each of the sample was assessed using the Reed Muench method. The mean viral titer is in different organs in the unvaccinated chromium supplemented group was lower as compared to the control (p<0.05) (FIG. 4).

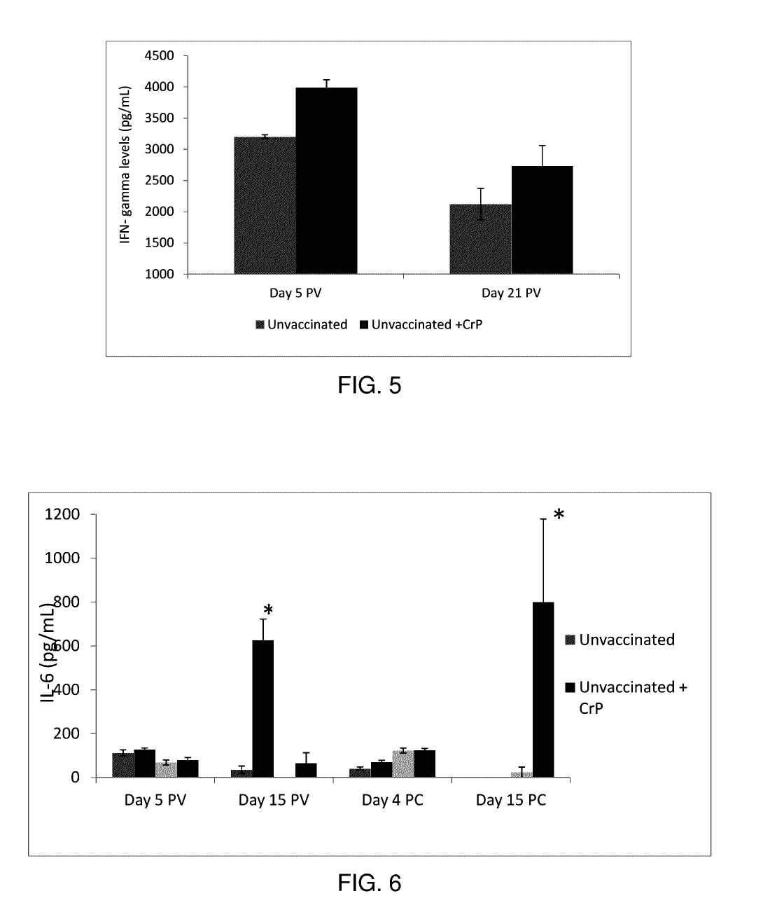

The serum profile of cytokines post-vaccination and challenge were measured by ELISA. Significantly higher (p<0.05) serum Interferon-γ (IFN-γ) levels were detected in the unvaccinated group upon supplementation of CrP for 5 days (FIG. 5).

Significantly higher (p<0.05) serum Interleukin-6 (IL-6) levels were observed in the unvaccinated group upon supplementation of CrP (FIG. 6). Post-challenge responses also showed an increasing trend for the CrP supplemented groups, as compared to the control groups.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

1. Anonymous, 2011. World Livestock Disease Atlas: A Quantitative Analysis of Global Animal Health Data A (2006-2009). In: The International Bank for Reconstruction and Development—The World Bank and The TAFS Forum, (Ed.), Washington, D.C.
2. OIE, 2012. Manual of Diagnostic Tests and Vaccines for Terrestrial Animals: Mammals, Birds and Bees, Biological Standards Commission. World Organization for Animal Health, Paris, pp. 1-19.
3. Talactac, M., Lee, J. S., Moon, H., Chowdhury, M. Y. E., and Kim, C. J. (2014) The antiviral effect of high molecular weight poly-gamma-glutamate against Newcastle disease virus on murine macrophage cells. Advanaces in Virology, Article ID 301386.
4. Elizondo-Gonzalez, R., Cruz-Suarez, L. E., Ricque-Marie, D., Mendoza-Gamboa, E., Rodriguez-Padilla, C., and Trejo-Avila. L. (2012) In vitro characterization of the antiviral activity of fucoidan from Cladosiphon okamuranus against Newcastle Disease Virus, Virol J.; 9: 307.
5. Ueda K., Kawabata R., Irie T., Nakai Y., Tohya Y., and Sakaguchi T. (2013) Inactivation of Pathogenic Viruses by Plant-Derived Tannins: Strong Effects of Extracts from Persimmon (Diospyros kaki) on a Broad Range of Viruses. PLoS ONE 8(1): e55343. doi:10.1371/journal.pone.0055343.

We claim:

1. A method of reducing viral load in poultry under viral challenge comprising the supplementation of the poultry with an efficacious amount of a composition comprising a chromium (III) salt of a carboxylic acid.

2. The method of claim 1, wherein the said composition forms low levels of Cr⁺⁶; and, said low levels of Cr⁺⁶ in said food composition having the equivalent of less than 8.3 parts per billion of Cr⁺⁶ per 8% total chromium in the food composition.

3. The method of claim 1, where the carboxylic acid is selected from the group consisting of short chain fatty acids.

4. The method of claim 3, where the carboxylic acid is more specifically propionic acid.

5. The method of claim 1, wherein the said viral challenge includes, but is not limited to, Newcastle disease, HPAI and LPAI.

6. The method claim 1, wherein the supplementation is through oral route.

7. The method of claim 1, wherein the efficacious dosage varies from 400 to 3000 ppm.

8. The method of claim 7, wherein the dosage is more precisely from 600 to 1500 ppm.

9. The method of claim 1, wherein reduction in viral load results in prevention of infection or reduction in clinical symptoms and mortality.

10. The method claim 9, wherein the prevention or control of infection results in enhanced health, performance and survival.
